(19) United States
(12) Patent Application Publication
(10) Pub. No.: US 2004/0023322 A1
(43) Pub. Date: Feb. 5, 2004

(54) Method of producing non-recombinant BMP-2 and use thereof

(76) Inventor: Clyde R. Goodheart, Lincolnshire, IL (US)

Correspondence Address: CLARK & ELBING LLP, 101 FEDERAL STREET, BOSTON, MA, 02110, US

(21) Appl. No.: 10/210,222

(22) Filed: Aug. 1, 2002

(51) Int. Cl. C12P 21/02; C12N 5/06

(52) U.S. Cl. 435/69.1; 435/325

(57) ABSTRACT

The invention features a method of producing non-recombinant BMP-2 by culturing mammalian cells that secrete BMP-2 and isolating the BMP-2 from the culture medium. The invention also features compositions containing purified BMP-2 and a method of using purified BMP-2 to promote bone formation.

### BACKGROUND OF THE INVENTION

The invention relates to the production and use of non-recombinant BMP-2.

Bone morphogenetic proteins (BMPs) are a subset of the transforming growth factor (TGF-β) superfamily of dimeric, disulfide crosslinked growth and differentiation factors. To date, at least six human BMPs have demonstrated osteogenic activity: BMP-2, -4, -5, -6, -7 (also referred to as osteogenic protein (OP-1)), and -8 (OP-2). In addition, a number of newly discovered molecules, including growth differentiation factors (GDFs) 1, 6, and 7 as well as BMP-9, dorsalis, and Vg1 can be considered to fall within the BMP family. While the BMPs are similar to other factors in the TGF-β superfamily, only BMPs have been demonstrated to induce either cartilage or bone formation in vivo. Specifically, BMP-2 has been found to be safe and feasible for use in the treatment of fractures and for use in bone regeneration. Recombinant human BMP-2 has also been extensively studied and has been found to demonstrate significant osteogenic activity in several models of bone formation.

The potential utility of BMP-2 has been widely recognized. It is contemplated that the availability of the pure BMP-2 protein would revolutionize orthopedic medicine, certain types of plastic surgery, and various periodontal and craniofacial reconstructive procedures. Currently, there are two major methods for the production of BMP-2. The first involves extracting the BMP-2 from demineralized cortical bone. The second method involves recombinant expression of BMP-2.

Both methods of production have significant drawbacks. The first method of purifying BMP-2 from bone is costly, time-consuming, and generally produces low yield. The second method enables production of BMP-2 with less cost and higher yield. There exists a need for a method of producing BMP-2 that combines the benefit of high yield and high activity.

### SUMMARY OF THE INVENTION

The invention features a method for producing non-recombinant BMP-2 by culturing mammalian cells that express BMP-2 and isolating BMP-2 from the culture. In some embodiments, the mammalian cells secrete BMP-2 into the culture medium, the culture medium is separated from the cells (i.e., the culture medium is rendered substantially free of cells), and BMP-2 is isolated from the culture medium. In other embodiments, the mammalian cells do not secrete BMP-2; here the cells are separated from the culture medium and BMP-2 is isolated from an extract of the cells. Desirably, the mammalian cells are human cells. More desirably, the mammalian cells are human non-cancer cells. The mammalian cells can be, e.g., stem cells, macrophages, fibroblasts (e.g., human fetal lung fibroblasts (e.g, MRC-5 cells (ATCC CCL-171), or MRC-9 cells), vascular cells, osteoblasts, chonidroblasts, osteoclasts, and osteocytes.

The invention also features a method for obtaining greater than 95% pure non-recombinant BMP-2 by culturing human non-cancer cells in culture medium, in which the cells express non-recombinant BMP-2, and purifying BMP-2 by chromatography such that BMP-2 is greater than 95% pure. In an embodiment, the human non-cancer cells secrete BMP-2 into the culture medium. In another embodiment, the culture medium containing BMP-2 is separated from the cells (i.e., such that the culture medium is rendered substantially free of cells), and the BMP-2 is purified from the culture medium. The human non-cancer cells can be stem cells, macrophages, fibroblasts (e.g., human fetal lung fibroblasts (e.g, MRC-5 cells (ATCC CCL-171), or MRC-9 cells), vascular cells, osteoblasts, chondroblasts, osteoclasts, and osteocytes.

The BMP-2 isolation methods of the invention include the steps of filtering the culture medium to produce a filtrate that contains BMP-2, followed by a purification step in which BMP-2 is purified from the filtrate using chromatography. The chromatography step can be performed by applying the filtrate to a first affinity column (e.g., a gelatin-sepharose column), in which some of the BMP-2 binds to the first affinity column and some BMP-2 is retained in the filtrate that passes through the first affinity column, referred to as the flow through. The BMP-2 that binds to the first affinity column is further processed by eluting the BMP-2 from the first affinity column by application of a first elution buffer which includes between 1 M and 10 M urea, between 10 mM and 50 mM 3-[cyclohexylamino]-1-propanesuflonic acid (CAPS) buffer, and is at a pH between 8.0 and 12.0. A first eluent containing the BMP-2 is collected and applied to a size exclusion column (e.g., a G-25, G-75, or G-100 column) to remove the urea and CAPS buffer. BMP-2 passes through the size exclusion column to produce a second filtrate, which is collected.

BMP-2 found in the flow through is further purified by applying the flow through to a second affinity column (e.g., an affinity column other than the gelatin-sepharose column; e.g., a heparin-sepharose column), so that BMP-2 binds to the second affinity column. BMP-2 is eluted from the second affinity column by application of a second elution buffer to the column, in which the second elution buffer includes between 0.1 M and 2 M NaCl, between 10 mM and 1 M Tris-HCl, and between 1 M and 10 M urea, and is at a pH between 5.0 and 10.0. Finally, BMP-2 is collected as a second eluent, which is then applied to a size exclusion column (e.g., a G-25, G-75, or G-100 column) to remove the urea. The second eluent passes through the size exclusion column to produce a third filtrate containing BMP-2, which is collected.

In an embodiment of the invention, the urea and CAPS buffer are present in the first elution buffer at 4 M and 50 mM, respectively, and the pH of the first elution buffer is 11.0. In another embodiment, the NaCl, Tris-HCl, and urea are present in the second elution buffer at 0.7 M, 50 mM, and 6 M, respectively, and the pH of the second elution buffer is 7.4.

In an embodiment of all features of the invention, the culture medium consists of medium 199 and can further contain 1.0 to 3.5 g/L bicarbonate salt, 1.0 to 5.0 g/L glucose, 10 to 30 μg/L dexamethasone, 1 to 10 g/L hydrolyzed protein (e.g., lactalbumin), and 5 to 15 μg/L insulin. The culture medium can also contain an antibiotic (e.g., penicillin), which can be present at a concentration of 50,000 to 200,000 units/L, and streplomycin, which can be present at a concentration of 0.05 to 0.2 g/L.

The invention also features a composition consisting of non-recombinant BMP-2 in which BMP-2 makes up greater than 95% of the composition and is capable of inducing bone formation when administered to a mammal.

The invention also features a composition for stimulating new bone formation in a patient in need thereof in which the composition consists of a pharmaceutically effective dose of substantially pure non-recombinant BMP-2, which can be administered to the patient. The BMP-2 is obtained by culturing human non-cancer cells, in culture medium in which BMP-2 is secreted into the culture medium, and purifying the BMP-2 from the culture medium.

The invention also features a method for stimulating new bone formation in a patient in need thereof in which a pharmaceutically effective dose of substantially pure non-recombinant BMP-2 is administered to the patient. The BMP-2 is obtained by culturing human non-cancer cells, in culture medium in which BMP-2 is secreted into the culture medium, and purifying the BMP-2 from the culture medium.

The invention also features a method for producing non-recombinant BMP-2 by culturing mammalian non-cancer cells in culture medium, in which the cells express and secrete BMP-2 into the culture medium, separating the culture medium from the cells, and purifying the BMP-2 from the culture medium.

The invention also features a method for producing non-recombinant BMP-2 by culturing mammalian non-cancer cells in culture medium, in which the cells express BMP-2, and purifying the BMP-2 from the culture medium or from the cells in the culture medium. In an embodiment, BMP-2 is purified from the cells by extraction from the cells.

In another embodiment, the BMP-2 composition also includes a matrix selected from the group consisting of fibrin, fibronectin, collagen, gelatin, agarose, a calcium phosphate containing compound (e.g., hydroxyapatite, tri-calcium phosphate, or amorphous calcium phosphate), a polymeric particle (e.g., poly(lactic acid), poly(glycolic acid), and copolymers of lactic acid and glycolic acid), an inorganic filler or particle (e.g., ceramic glass, porous ceramic particles or powders, mesh titanium or titanium alloy, particulate titanium or titanium alloy, or bioglass), and combinations thereof. The composition can be administered in a solid, paste, gel, or liposome formulation.

The composition can additionally include a growth factor selected from the group consisting of insulin-like growth factor (IGF)-I, IGF-II, fibroblast growth factor (FGF), growth hormone (GH), platelet-derived growth factor (PDGF)-I, PDGF-II, interleukin (IL)-1, transforming growth factor (TGF)-α, TGF-β, epidermal growth factor (EGF), tumor necrosis factor (TNF), vascular endothelial growth factor (VEGF), and nerve growth factor (NGF). In another embodiment, the matrix can be selected from the group consisting of fibrin, fibronectin, collagen, gelatin, agarose, a calcium phosphate containing compound (e.g., hydroxyapatite, tri-calcium phosphate, or amorphous calcium phosphate), a polymeric particle (e.g., poly(lactic acid), poly(glycolic acid), and copolymers of lactic acid and glycolic acid), an inorganic filler or particle (e.g., ceramic glass, porous ceramic particles or powders, mesh titanium or titanium alloy, particulate titanium or titanium alloy, or bioglass), and combinations thereof. In yet another embodiment, the composition is administered in a solid, paste, gel, or liposome formulation.

The invention takes advantage of the discovery that mammalian cells (e.g., human, non-cancer cells) produce and secrete biologically active BMP-2 into culture medium. It is believed that the secreted BMP-2 has greater biological activity than recombinalatly-expressed BMP-2. Further, secreted BMP-2 is easier to purify than BMP-2 extracted from bone.

Definition

By “substantially pure” is meant a protein or polypeptide which has been separated from components which naturally accompany it. Typically, a protein of interest is substantially pure when at least 60% to 75% of the total protein in a sample is the protein of interest. Minor variants or chemical modifications typically share the same polypeptide sequence. A substantially pure protein will typically comprise over about 85 to 90% of the protein in the sample, more usually will comprise at least about 95%, and preferably will be over about 99% pure. Normally, purity is measured on a chromatography column, polyacrylamide gel, or by HPLC analysis.

### BRIEF DESCRIPTION OF THE DRAWINGS

FIG. 1 is a photograph of a silver-stained gel showing the presence of human BMP-2 following purification of BMP-2 from the culture medium of MRC-5 cells. BMP-2 was harvested from culture medium that was incubated with MRC-5 cells for 3 days and filtered through a fiberglass filter to remove cell debris. The filtrate was pumped through a gelatin-sepharose column and the collected fractions with the highest protein concentration were added to a G-100 column. Nine fractions were collected from the G-100 column. The fractions were reduced using β-mercaptoethanol, loaded onto a 12% gel and resolved by electrophoresis. Lane 1: Recombinant BMP-2, which exhibits a molecular weight of ˜15 kDa and corresponds to the monomeric species of BMP-2. Lanes 2-9: Fractions 2-9 were collected from the G-100 column. The lower band in lanes 3-6 represents monomeric BMP-2 with a MW of ˜24 kDa. Lane 10: Pre-stained molecular weight marker (Pierce, Rockford Ill.; product number 26691): Myosin—223 Kd, phosphorylated B—111 Kd, BSA—81.7 Kd, Ovalbumini—47.9 Kd, carbonic anhydrase—31.6 Kd, trypsin inhibitor—24.4 Kd, and lysozyme—15.6 Kd.

FIG. 2 is a photograph of a western blot showing the presence of human BMP-2 following purification from the culture medium of MRC-5 cells. BMP-2 is resolved by SDS-PAGE on a 12% reducing gel, transferred to a PVDF membrane, exposed to a primary BMP-2 antibody (Sigma-Aldrich Cat. No. B-9953) and a secondary anti-murine antibody conjugated to horse radish peroxidase (Sigma-Aldrich Cat. No. A-6782), and stained using 3,3′-diaminobenzidine (DAB). Lane 1: Recombinant BMP-2 with an apparent molecular weight of ˜15 kDa, which corresponds to the monomeric species of BMP-2. Lanes 2-4: Aliquots of fractions 3, 4, and 5 from FIG. 1 (see lanes 3, 4, and 5) showing BMP-2 with a molecular weight of ˜85 kDa. BMP-2 that resolves at ˜50-60 kDa is also apparent in lanes 3 and 4. Lanes 5-7: Peak fractions containing BMP-2 that were eluted from a G-100 column (using the same method described in FIG. 1) from a second harvest of MRC-5 cell culture medium. The higher molecular weight band (most visible in lane 6) represents BMP-2 with an apparent molecular weight of ˜112 kDa. Lane 8: BMP-2-containing fractions from a third experiment in which the three peak fractions eluted from the gelatin-sepharose column were pooled, reduced, and loaded onto the gel. No BMP-2 was observed in the fractions generated in this experiment. Lane 9: Culture medium containing BMP-2 was loaded onto a gelatin-sepharose column and the flow through was collected. The flow through was loaded onto a heparin-sepharose column (Amersham BioScience Cat. No. 17-0998-01) and eluted with elution buffer (NaCl 0.7 M, Tris-HCl 50 mM, urea 6 M, pH 7.4) to produce an eluent. The two peak fractions from the eluent were pooled and a sample of the fractions was loaded into lane 9. BMP-2 appears as a band at ˜112 kDa and at ˜37 kDa. Lane 10: Pre-stained molecular weight marker (see FIG. 1).

### DETAILED DESCRIPTION

The invention features BMP-2 produced by mammalian cells (e.g., human, non-cancer cells), which is secreted into culture medium surrounding the cells, and can be purified using standard column chromatography. Surprisingly, secreted BMP-2 purified from culture medium exhibits greater biological activity than recombinantly-produced BMP-2. Furthermore, BMP-2 isolated by the methods disclosed herein is easier to produce than the prior art-disclosed methods of extracting BMP-2 from bone.

Another advantage of BMP-2 produced by the methods disclosed herein is that the protein, once purified, retains its native conformation and does not precipitate. In contrast to the prior art-produced BMP-2, the BMP-2 produced herein also does not require the use of harsh chemical agents (e.g., guanidine hydrochloride) for extraction of the protein, and no refolding is necessary after purification and prior to use.

In addition, the use of a mammalian cell that can be grown in culture enables the production and purification of BMP-2 in large quantities using mass culture techniques. Furthermore, because the invention encompasses the use of a cell line of human origin, the production and purification of BMP-2 from these cells will be more structurally similar (e.g., with respect to disulfide crosslinks, glycosylation, and post-translation modification) to BMP-2 naturally found in human subjects, and the BMP-2 will be less likely to elicit immune reactions from human subjects administered the BMP-2-containing composition.

BMP-2

Tissue culture cells are typically grown in the laboratory in a closed culture system that requires replacement of the medium after growth of the cells for several days. This replacement replenishes the nutrients required for the cells to grow. The production of BMP-2 utilizes two different types of culture medium: a growth culture medium and a production culture medium. The growth culture medium is used to expand the BMP-2 expressing cells and does not promote the production of significant amounts of BMP-2. The production culture medium is used to promote expression and secretion of BMP-2 by the BMP-2 producing cells.

The present invention provides methods for purifying cell-produced and secreted BMP-2. The method involves incubating BMP-2 cells in production culture medium for a period of time sufficient to allow the cells to produce and secrete BMP-2 into the medium (e.g., 8 hours, 1 day, 3 days, or 1 week). The production culture medium is collected approximately 2-3 times per week and BMP-2 is purified from the collected medium. BMP-2 can also be collected from the BMP-2-expressing cells by extracting the protein from the cells. Examples of mammalian cells that express and secrete BMP-2 into culture medium include stem cells, macrophages, fibroblasts (e.g., human fetal lung fibroblasts (e.g, MRC-5 cells (ATCC CCL-171), or MRC-9 cells), vascular cells, osteoblasts, chondroblasts, osteoclasts, and osteocytes. The mammalian cells can: be obtained from primary cultures (e.g., foreskin fibroblasts isolated directly from patient tissue), or from established cultures (e.g., cells purchased from the American Type Culture Collection (ATCC)). The cells are first suspended in a growth culture medium containing serum (e.g., fetal bovine serum). The cells are incubated in the growth culture medium to form a monolayer of cells. The cells are cultured for 1 day up to 1 week, depending on the cell type, confluency, and growth properties of the cells. Once the cells have been sufficiently expanded, the growth culture medium is replaced by a production culture medium (e.g., Medium 199) lacking serum, thereby promoting the production of BMP-2. The cells are cultured for approximately six weeks.

The production culture medium, which contains the secreted BMP-2, is harvested 2-3 times per week, and replaced with fresh production culture medium. After approximately six weeks, the production culture medium is replaced with growth culture medium. After a brief incubation period, the culture medium is removed, the cells are rinsed with a solution of ethylenediamine tetraacetic acid (EDTA) followed by a brief treatment with a fresh trypsin solution, and the cells are gently removed from the culture container. A portion of the trypsin-treated cells are then transferred to a second container containing fresh growth culture medium. The cells are again allowed to grow into a monolayer and the process is repeated.

The conditioned culture medium is clarified by filtration through a course fiberglass filter. The clarified culture medium is then generally exposed to an affinity chromatography column and BMP-2 is eluted from the column using an elution buffer. The BMP-2 can then be dialyzed into the desired buffer or a size-exclusion column can be used to exchange the buffer. Finally, the purified BMP-2 is lyophilized.

In addition, BMP-2 can also be extracted directly from the cells. If this technique is used, it is preferable that the cells used are noni-cancer cells. The cells can be cultured, as described above, and after a sufficient length of time (e.g., 8 hours, 1 day, 3 days, or 1 week), the cells can be separated from the culture medium and lysed using any one of several methods known in the art (see, for example, Gilbert et al., J. Immunol. Methods 261:85-101, 2002; Dudani et al., Thromb. Res. 69:185-96, 1993; and Schutte et al., Biotechnol. Appl. Biochem. 12:599-620, 1990). Once an extract has been produced, BMP-2 released from the cells can be purified by column chromatography, as described below.

BMP-2 purified by the methods of the invention exhibits a molecular weight of between 24 and 112 kDa. In the absence of studies on the post-translational modification of BMP-2 produced by the cells of the invention, it appears that BMP-2 that exhibits a molecular weight of about 24 to 37 kDa represents the monomeric form. BMP-2 that exhibits a molecular weight of about 60 to 78 kDa represents the dimeric form. BMP-2 that exhibits a molecular weight of about 87 kDa represents the trimeric form. BMP-2 that exhibits a molecular weight of about 112 kDa represents a multimeric form.

Cell Types for Producing BMP-2

BMP-2 can be produced and isolated according to the methods disclosed herein using a variety of cell types. In particular, BMP-2 is produced and secreted into culture medium by stem cells, macrophages, fibroblasts (e.g., human fetal lung fibroblasts (e.g, MRC-5 cells (ATCC CCL-171), or MRC-9 cells), vascular cells, osteoblasts, chonidroblasts, osteoclasts, osteocytes, and cancer cell types (e.g., human osteosarcoma cells (HOS)). Any culture medium that supports the growth of these cells is suitable for use in the methods of the invention. Desirable culture medium is described herein (e.g., production culture medium versus growth culture medium). Additional constituents can be added to the culture medium to enhance expression and secretion of BMP-2 from the cells including, for example, serum (e.g., fetal bovine serum), theophyllin, retinoic acid, and calcium ions.

Uses of BMP-2

Purified BMP-2 described herein can be administered to augment bone growth, to prevent bone loss due to diseases or disorders (e.g., osteoporosis, osteogenesis imperfecta, and errors in development), to speed fracture healing and bone repair, to facilitate bone repair and reconstruction (due to, for example, cancer surgery), to improve bone grafting, to speed healing of traumatic fractures, to augment bonding of resected bone surfaces to porous, biocompatible prostheses, and to effect repair of non-uniform fractures.

In addition to treating human patients, BMP-2 can also be administered for veterinary applications. Depending on the particular veterinary application, BMP-2 can be produced in a cell type that corresponds to the animal that is to be treated. For example, it is envisioned that BMP-2 can be produced and secreted from, e.g., canine, feline, bovine, or equine cells and the BMP-2 purified from these cells can be used in applications specific for, e.g., dogs, cats, cows, or horses.

BMP-2 can be used either alone or in combination with biodegradable materials or pharmaceutical carriers (see below). In treating humans and animals, progress may be monitored by periodic assessment of bone growth and/or repair using, for example, x-rays.

The present methods and compositions may also have prophylactic uses in closed and open fracture reduction and also in the improved fixation of artificial joints. The invention is applicable to stimulating bone repair in congenital, trauma-induced, or oncologic resection-induced defects, and also is useful in the treatment of periodontal disease and other tooth repair processes, and even in cosmetic plastic surgery.

Matrix Materials

Isolated BMP-2 produced by the methods of the invention can be combined with any suitable matrix material for administration to a patient. Suitable matrix material includes, for example, fibrin, fibronectin, collagen (see e.g., U.S. Pat. No. 4,394,370), gelatin, agarose, a calcium phosphate containing compound (e.g., hydroxyapatite, tri-calcium phosphate, amorphous calcium phosphate, and other calcium phosphate compounds), a polymeric particle (e.g., poly(lactic acid), poly(glycolic acid), and copolymers of lactic acid and glycolic acid), an inorganic filler or particle (e.g., ceramic glass, porous ceramic particles and powders, mesh titanium and titanium alloy, particulate titanium and titanium alloy, and bioglass), and combinations thereof.

A biodegradeable matrix of porous particles for delivery of an osteogenic protein is disclosed in U.S. Pat. No. 5,108,753. A slow release delivery system that can be used with BMP-2 is described in U.S. Pat. No. 5,108,753. Okada et al., U.S. Pat. Nos. 4,652,441; 4,711,782; 4,917,893; and 5,061,492; and Yamamoto et al., U.S. Pat. No. 4,954,298 disclose other prolonged-release compositions that can be used with BMP-2 in the methods of the invention.

The choice of matrix material will differ according to the particular circumstances and the site of the bone that is to be treated. Matrices such as those described in U.S. Pat. No. 5,270,300 and U.S. Pat. No. 5,763,416 may be employed. Physical and chemical characteristics, such as, e.g., biocompatibility, biodegradability, strength, rigidity, interface properties and even cosmetic appearance may be considered in choosing a matrix, as is well known to those of skill in the art. Appropriate matrices will both deliver the BMP-2 composition and also provide a surface for new bone growth, i.e., the matrix will act as an in situ scaffolding through which bone progenitor cells may migrate.

A particularly important aspect of the present invention is its use in connection with orthopedic implants, interfaces, and artificial joints, including implants themselves and functional parts of an implant, such as, e.g., surgical screws, pins, and the like. In preferred embodiments, it is contemplated that the metal surface or surfaces of an implant or a portion thereof, such as a titanium surface, can be coated with a matrix material admixed with the BMP-2 composition, e.g., hydroxyapatite, and then used in the methods of the invention.

In certain embodiments, non-biodegradable matrices may be employed, such as sintered hydroxyapatite, bioglass, aluminates, other bioceramic materials, and metal materials, particularly titanium. A suitable ceramic delivery system is that described in U.S. Pat. No. 4,596,574. Polymeric matrices may also be employed, including acrylic ester polymers and lactic acid polymers, as disclosed in U.S. Pat. Nos. 4,526,909 and 4,563,489, respectively.

In preferred embodiments, it is contemplated that a biodegradable matrix will likely be most useful. A biodegradable matrix is generally defined as one that is capable of being resorbed into the body. Potential biodegradable matrices for use in connection with the compositions, devices, and methods of this invention include, for example, biodegradable and chemically defined calcium sulfate, tri-calcium phosphate, hydroxyapatite, polylactic acid, polyanhydrides, matrices of purified proteins, and semi-purified extracellular matrix compositions. The most preferred matrices are those prepared from tendon or dermal collagen, as may be obtained from a variety of commercial sources, such as, e.g., Sigma and Collagen Corporation. Collagen matrices may also be prepared as described in U.S. Pat. Nos. 4,394,370 and 4,975,527. Currently, the most preferred collagenous material is that termed ULTRAFIBER™, obtainable from Norian Corp. (Mountain View, Calif.).

Other natural and synthetic matix compositions suitable for use in the invention are disclosed in, for example, U.S. Pat. Nos. 6,398,816; 5,597,897; 5,385,887; 5,788,959; 5,631,142; 5,782,971; and 6,027,743.

Bioactive Molecules

Several other useful bioactive molecules can be prepared with BMP-2 in a pharmaceutical composition or admixed in a matrix material and administered to a subject for the purpose of promoting bone formation, growth, and healing. Examples of these bioactive molecules include growth factors, morphogenesis factors, structural proteins, or cytokines that enhance the temporal sequence of wound repair, alter the rate of proliferation, increase the metabolic synthesis of extracellular matrix proteins, or direct phenotypic expression in endogenous cell populations. Representative proteins include other bone growth factors (BMPs, insulin-like growth factors (IGF)-I and IGF-II) for bone healing, cartilage growth factors (CGF, transforming growth factor (TGF)-α, and TGF-β) for cartilage healing, nerve growth factors (NGF) for nerve healing, and general growth factors important in wound healing, such as platelet-derived growth factor (PDGF (e.g., PDGF-I and PDGF-II)), vascular endothelial growth factor (VEGF), keratinocyte growth factor (KGF), endothelial derived growth supplement (EDGF), epidermal growth factor (EGF), fibroblast growth factor (FGF) for wound and skin healing, and other factors, including, for example, interleukin-1 (IL-1) and tumor necrosis factor (TNF).

It is well established that certain bioactive molecules can induce formation of bone or connective tissue. In addition to members of the TGF-β superfamily (e.g., BMP-2-15, TGF-α, TGF-β, and IGF), other osteoinductive factors can also be included in a composition administered to a subject for promoting bone formation, growth, and repair, such as other BMPs (e.g., BMP-3, BMP-4, BMP-5, BMP-6, BMP-7, BMP-8, BMP-9, BMP-10, BMP-11, BMP-12, BMP-13, BMP-15, BMP-16, BMP-17, and BMP-18), skeletal growth factor (SGF), osteoblast-derived growth factors (ODGFs), retinoids, growth hormone (GH), and transferrin.

Pharmaceutical Compositions and Dosages

BMP-2 produced using the methods of the invention may be administered to a patient for in vivo therapy by any method known to one skilled in the art. BMP-2 may be admixed, encapsulated, conjugated, or otherwise associated with other molecules, molecule structures or mixtures of compounds, as for example, liposomes or other formulations. BMP-2 may be administered in a solid, paste, or gel formulation. BMP-2 may be administered in the form of a pharmaceutically acceptable salt, ester, or salt of such ester, or any other compound which, upon administration to an animal, including a human, is capable of providing (directly or indirectly) the biologically active metabolite or residue thereof.

Methods well known in the art for making formulations are found, for example, in Remington's Pharmaceutical Sciences (18th edition), ed. A. Gennaro, 1990, Mack Publishing Company, Easton, Pa. The amount of active ingredient in the compositions of the invention can be varied. One skilled in the art will appreciate that the exact individual dosages may be adjusted somewhat depending upon a variety of factors, including the time of administration, the route of administration, the nature of the formulation, the nature of the subject's conditions, and the age, weight, health, and gender of the patient. Generally, dosage levels of between 0.1 μg/kg to 100 mg/kg of body weight are administered daily as a single dose or divided into multiple doses. Desirably, the general dosage range is between 250 μg/kg to 5.0 mg/kg of body weight per day. Variations in these dosage levels can be adjusted using standard empirical routines for optimization, which are well known in the art. In general, the precise therapeutically effective dosage will be determined by the attending physician in consideration of the above identified factors.

The candidate compound of the invention can be administered in a sustained release composition, such as those described above or in, for example, U.S. Pat. No. 5,672,659 and U.S. Pat. No. 5,595,760. The use of immediate or sustained release compositions depends on the type of condition being treated. If the condition consists of an acute or over-acute disorder, a treatment with an immediate release form will be desired over a prolonged release composition. Alternatively, for preventative or long-term treatments, a sustained released composition will generally be desired.

The amount of BMP-2 that is applied in a pharmaceutical composition or in the form of a matrix and/or the amount of BMP-2/matrix material that is applied to the bone tissue will be determined by the attending physician or veterinarian considering various biological and medical factors. For example, one would wish to consider the particular matrix, the amount of bone weight desired to be formed, the site of bone damage, the condition of the damaged bone, the patient's or animal's age, sex, and diet, the severity of any infection, the time of administration, and any further clinical factors that may affect bone growth, such as serum levels of various factors and hormones. The suitable dosage regimen will therefore be readily determinable by one of skill in the art in light of the present disclosure, bearing in mind the individual circumstances

The following examples are meant to illustrate the principles and advantages of the present invention. They are not intended to be limiting in any way.

### EXAMPLE 1

Isolation and Purification BMP-2

MRC-5 cells were obtained from American Type Culture Collection (ATCC; CCL-171) and cultured in the laboratory in a culture medium containing penicillin and streptomycin at a concentration of 10,000 units/mL and 10 mg/mL, respectively. The cells were passaged by brief treatment with a trypsin solution (0.25% of trypsin 1:250 in phosphate buffered saline) containing 0.02% EDTA (ethyleenediamine tetraacetic acid and no calcium or magnesium salts) at room temperature for 45 minutes to dissociate the cells form the tissue culture dish. The cells were resuspended in 20 mL of MCDB 105 culture medium supplemented with fetal bovine serum (5%). The resuspended cells were then placed in petri dishes or flasks and incubated at 36° C. The cells were expanded in growth culture medium, which was changed after incubation for about one week to ten days (i.e., after the cells had formed a complete monolayer). It is possible, however, to incubate the cells for a much shorter or longer time period, e.g. for one day, or for up to several weeks. The cells were passaged by removing the cells from the surface of the culture vessel by rinsing first with EDTA solution (described above) and then treating with the trypsin solution described above. The cells were transferred to plastic roller bottles (Becton Dickinson) at a split ratio of 4:1 based on relative surface areas, using 100 mL/bottle of the same growth medium described above. In five more days of incubation, the cells had completely covered the surface of the roller bottle, and were split once more as described above.

After the cells had again formed a confluent sheet, the growth culture medium was replaced with production culture medium (100 mL/bottle). This medium was standard Medium 199, obtained from a commercial source as a dry powder and reconstituted with tissue-culture-grade distilled water according to the manufacturer's instructions. The following ingredients were added to the standard Medium 199: lactalbumin hydrolysate 5 g/L; sodium bicarbonate, 2.2 g/L; HEPES buffer, free acid, 0.794 g/L; HEPES buffer, sodium salt, 1.735 g/L; penicillin 100,000 units/L; streptomycin 0.1 g/L; glucose 3 g/l; insulin 10 mg/L; and dexamethasone 20 g/L. Variations on the medium are contemplated.

We have obtained optimal, high-level cellular BMP-2 production in MRC-5 cells grown in tissue culture medium fortified with a number of components, as follows: (a) 1.0 to 3.5 g/L bicarbonate salt; (b) 1.0-5.0 g/L glucose; (c) 10-30 mg/L dexamethasone; (d) 1-10 g/L hydrolyzed protein; and (e) 5-15 mg/L insulin. In preferred embodiments, the culture medium further contains penicillin and/or streptomycin (e.g., 50,000-200,000 units/L penicillin and 0.05 to 0.2 g/L streptomycin), and also contains 5-25 mmol/L HEPES buffer, to give a pH of 6.8 to 7.9.

The culture medium was left on the cells for a time sufficient to eliminate fetal bovine serum remaining from the growth medium, i.e., 2 hours to about 2 days. The culture medium was then discarded and replaced with fresh production culture medium. This medium was harvested from the cells by pouring it off every two to three days, and replaced with fresh production culture medium. The cells remained in the container after each harvest of conditioned culture medium.

Because of the well known susceptibility of cellular proteins to degradation by proteases, the BMP-2 was purified from each batch of conditioned culture medium harvested from the cells as soon as possible on the same day it was harvested, using the following procedure that permitted purification in minimum time. The production culture medium was filtered through a fiberglass filter to remove any cells or cell debris that might be present, and the filtrate was pumped through an affinity chromatography column containing a bed of gelatin-sepharose (Amersham Biosciences, Cat. No. 17-0956-01). Because some of the BMP-2 did not bind to the gelatin-sepharose, the flow through was saved and BMP-2 was subsequently purified from the flow through (see below). The column bed was flushed with an equilibration buffer (dihydrogen sodium phosphate (Na₂HPO₄) 10 mmol/L, sodium chloride (NaCl) 150 mmol/L, pH 7.2) until the absorption at 280 nm had returned to baseline. Elution buffer (50 mmol/L 3-[cyclohexylamino]-1-propanesulfonic acid (CAPS) buffer, 4 mol/L urea, pH 11.0) was pumped through the column to elute the BMP-2 from the affinity column material. A single sharp absorptive peak was collected and the fractions containing the peak were pooled.

BMP-2 present in the flow through was purified by adding the flow through to a heparinsepharose affinity chromatography column (Amersham Bioscience catalog #17-0998-01). The column bed was flushed with an equilibration buffer (dihydrogen sodium phosphate (Na₂HPO₄) 10 mmol/L, sodium chloride (NaCl) 150 mmol/L, pH 7.2) until the absorption at 280 nm had returned to baseline. A second elution buffer (NaCl 0.7 M, Tris-HCl 50 mM, urea 6 M, pH 7.4) was pumped through the column to elute the BMP-2 from the affinity column material. A single sharp absorptive peak was collected and the fractions containing the peak were pooled.

The peak fractions containing BMP-2 from the gelatin-sepharose and the heparinsepharose affinity columns were then separately passed through either a G-25, G-75, or G-100 column equilibrated with water to remove any urea or CAPS buffer present in the elution buffers. A broad peak was collected containing BMP-2. This was filtered through a sterile 0.2 μm filter for sterilization and lyophilized. As an alternative to the G-25, G-75, or G-100 column, the peak fractions containing BMP-2 can also be dialyzed against a phosphate buffer (0.05 mol/L Na₂HPO₄, pH 7.5, and 0.1 mol/L NaCl) to remove any urea or CAPS buffer.

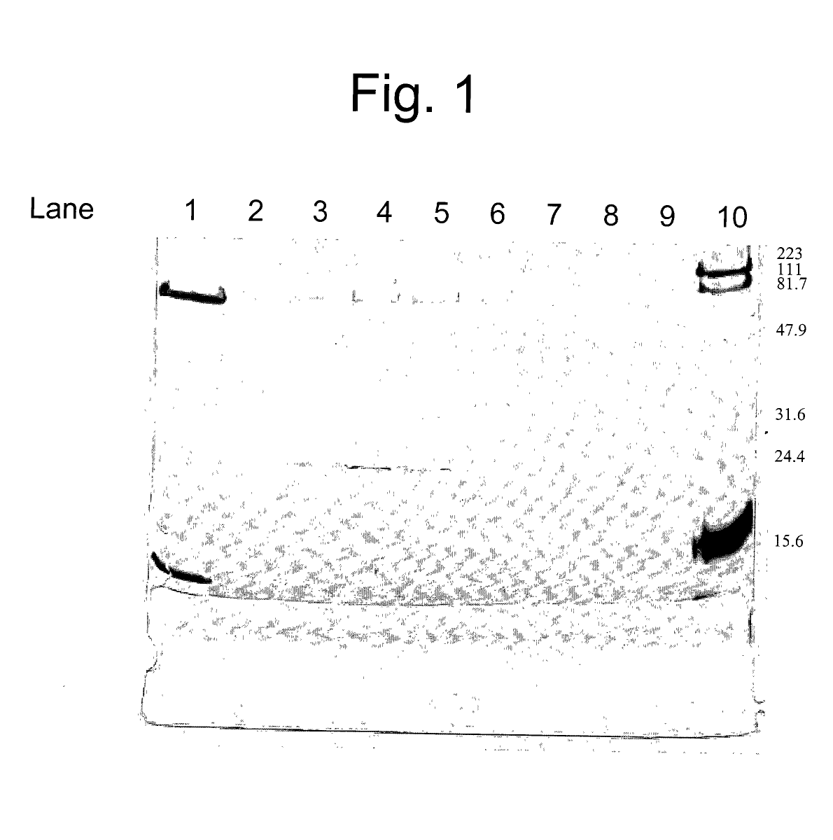

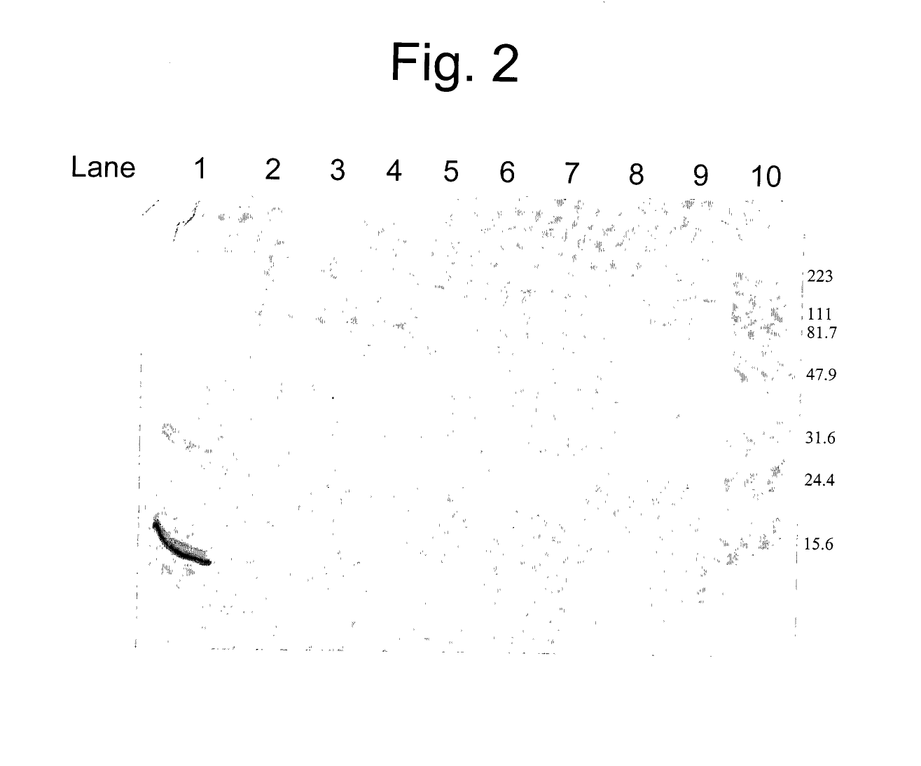

Samples taken during the procedure and after lyophilization were analyzed by sodium dodecyl sulfate-polyacrylamide gel electrophoresis (SDS-PAGE) under both reducing and non-reducing conditions. BMP-2 was reduced using either dithiothreitol (DTT) or β-mercaptoethanol (βME), however βME was more effective at reducing BMP-2. The pattern of bands on the gels indicated that the product was BMP-2 and that it had been isolated to a high degree of purity (i.e., >95% purity; see FIGS. 1 and 2). The reduced BMP-2 demonstrated a molecular weight of ˜24 kDa (FIG. 1). BMP-2 also demonstrated a molecular weight of ˜85 kDa (trimer) and ˜112 kDa (multimers) (FIG. 2).

### EXAMPLE 2

Biological Activity Test—Induction of Alkaline Phosphatase

BMP-2 isolated and purified by the method described in Example 1 can be tested for biological ability by the ability to induce alkaline phosphatase (ALP).

The ability of purified BMP-2 to promote bone growth can be assayed using a quantitative in vitro assay which is both rapid and sensitive (Jortikka et al., Life Sciences 62:2359-2368, 1998). This assay measures the conversion of a skeletal muscle myoblast cell to an osteoblast-like cell. The assay utilizes the skeletal muscle cell line C2C12. This cell line is readily available from the ATCC. C2C12 is a mouse myoblast cell line that has been shown to convert its differentiation pathway from muscle cell (myoblast) to bone cell (osteoblast) in the presence of BMP-2 (Katagiri et al., J Cell. Biol. 127:1755-1766, 1994). BMP-2 is incubated with the C2C12 cells for a sufficient time (up to 14 days) to permit differentiation into osteoblast-like cells.

Each 2 cm² well of a 24-well plate can be seeded with 5×10⁴ C2C12 cells. BMP-2 is placed in the wells followed by the addition of growth medium. The wells are then seeded with the cells and incubated to permit differentiation and ALP production. Recombinant human BMP-2 can be used as a positive control. Growth medium can be changed every 3-5 days. After 13-15 days, the cells can be lysed, sonicated, and the supernatant assayed for ALP enzymatic activity. The production of ALP is an indication that the purified BMP-2 is biologically active.

ALP activity of purified BMP-2 can also be determined in a similar manner using the method of Luben, Wong and Cohn (Endocrinology 35:778, 1983) with n-nitro-phenylphosphate as the substrate.

### EXAMPLE 3

Biological Activity Test—Induction of Ectopic Bone Formation

BMP-2 isolated and purified by the method described in Example 1 can be tested for the ability to induce ectopic bone formation. An assay for the ability of BMP-2, derived by the methods of the invention, to induce bone formation may be conducted using the bone induction bioassay described by Sampath & Reddi (Proc. Natl. Acad. Sci. USA 78:7599-7603, 1981). This is a rat bone formation assay that is routinely used to evaluate the osteogenic activity of bone inductive factors.

Other Embodiments

All publications and patent applications mentioned in this specification are herein incorporated by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth.

What is claimed is:

1. A method for producing non-recombinant bone morphogenetic protein-2 (BMP-2), said method comprising the steps of:
(a) culturing mammalian cells that express BMP-2; and
(b) isolating BMP-2 from said culture.

2. The method of claim 1, wherein said cells are non-cancer cells.

3. The method of claim 1, wherein said cells secrete said BMP-2 into the culture medium.

4. The method of claim 3, wherein said culture medium comprises medium 199.

5. The method of claim 4, wherein said medium further comprises 1.0 to 3.5 g/L bicarbonate salt; 1.0 to 5.0 g/L glucose; 10 to 30 μg/L dexamethasone; 1 to 10 g/L hydrolyzed protein; and 5 to 15 μg/L insulin.

6. The method of claim 5, wherein said medium further comprises an antibiotic.

7. The method of claim 6, wherein said antibiotic is penicillin, which is present at a concentration of 50,000 to 200,000 units/L, and streptomycin, which is present at a concentration of 0.05 to 0.2 g/L.

8. The method of claim 5, wherein said hydrolyzed protein comprises lactalbumin hydrolysate.

9. The method of claim 1, wherein said mammalian cells are human cells.

10. The method of claim 9, wherein said human cells are non-cancer cells.

11. The method of claim 1, wherein said mammalian cells are selected from stem cells, macrophages, fibroblasts, vascular cells, osteoblasts, chondroblasts, osteoclasts, and osteocytes.

12. The method of claim 11, wherein said fibroblasts are human fetal lung fibroblasts.

13. The method of claim 12, wherein said human fetal lung fibroblasts are selected from MRC-5 cells and MRC-9 cells.

14. The method of claim 3, wherein the method comprises the steps of:
(i) filtering said culture medium to produce a filtrate comprising said BMP-2; and
(ii) purifying said BMP-2 from said filtrate using chromatography.

15. The method of claim 13, wherein said chromatography comprises the steps of:
(1) applying said filtrate to a first affinity column, wherein said filtrate passes through said first affinity column to produce a flow through and wherein some of said BMP-2 in said filtrate binds to said first affinity column;
(2) eluting said bound BMP-2 from said affinity column by applying a first elution buffer comprising between 1 M and 10 M urea and between 10 mM and 50 mM 3-[cyclohexylamino]-1-propanesulfonic acid (CAPS) buffer, wherein said first elution buffer is at a pH between 8.0 and 12.0;
(3) collecting a first eluent comprising said BMP-2;
(4) applying said first eluent to a size exclusion column to remove said urea and CAPS buffer, wherein said BMP-2 passes through said size exclusion column to produce a second filtrate; and
(5) collecting said second filtrate comprising said BMP-2.

16. The method of claim 15, wherein said method further comprises the steps of:
(6) applying said flow through comprising said BMP-2 to a second affinity column, wherein said BMP-2 binds to said second affinity column;
(7) eluting said BMP-2 from said second affinity column by applying a second elution buffer comprising between 0.1 M and 2 M NaCl, between 10 mM and 1 M Tris-HCl, and between 1 M and 10 M urea, wherein said second elution buffer is at a pH between 5.0 and 10.0;
(8) collecting a second eluent comprising said BMP-2;
(9) applying said second eluent to a size exclusion column to remove said urea, wherein said BMP-2 passes through said size exclusion column to produce a third filtrate; and
(10) collecting said third filtrate comprising said BMP-2.

17. The method of claim 15, wherein said first affinity column is a gelatin-sepharose column.

18. The method of claim 16, wherein said second affinity column is a heparin-sepharose column.

19. The method of claim 15, wherein said urea is present at a concentration of about 4 M, said CAPS buffer is present at a concentration of about 50 mM, and said pH is about 11.0.

20. The method of claim 16, wherein said NaCl is present at a concentration of about 0.7 M, said Tris-HCl is present at a concentration of about 50 mM, said urea is present at a concentration of about 6 M, and said pH is about 7.4.

21. The method of claim 15 or 16, wherein said size exclusion column is a G-100, a G-75, or a G-25 column.

22. The method of claim 15 or 16, wherein after said method is performed, said BMP-2 is greater than 95% pure by weight.

23. The method of claim 1, wherein said BMP-2 is capable of inducing bone formation when administered to a mammal.

24. A method for producing non-recombinant BMP-2 said method comprising the steps of:
(a) culturing mammalian cells that secrete non-recombinant BMP-2 in a culture medium; and
(b) isolating said BMP-2 from said medium.

25. The method of claim 24, wherein said mammalian cells secrete said BMP-2 into culture medium.

26. The method of claim 26, wherein said culture medium comprises medium 199.

27. The method of claim 26, wherein said medium further comprises 1.0 to 3.5 g/L bicarbonate salt; 1.0 to 5.0 g/L glucose; 10 to 30 μg/L dexamethasone; 1 to 10 g/L hydrolyzed protein; and 5 to 15 μg/L insulin.

28. The method of claim 27, wherein said medium further comprises an antibiotic.

29. The method of claim 28, wherein said antibiotic is penicillin, which is present at a concentration of 50,000 to 200,000 units/L, and streptomycin, which is present at a concentration of 0.05 to 0.2 g/L.

30. The method of claim 27, wherein said hydrolyzed protein comprises lactalbumin hydrolysate.

31. The method of claim 24, wherein said mammalian cells are selected from stem cells, macrophages, fibroblasts, vascular cells, osteoblasts, chondroblasts, osteoclasts, and osteocytes.

32. The method of claim 31, wherein said fibroblasts are human fetal lung fibroblasts.

33. The method of claim 32, wherein said human fetal lung fibroblasts are selected from MRC-5 cells and MRC-9 cells.

34. The method of claim 24, wherein the method comprises the steps of:
(i) filtering said culture medium to produce a filtrate comprising said BMP-2; and
(ii) purifying said BMP-2 from said filtrate using chromatography.

35. The method of claim 34, wherein said chromatography comprises the steps of:
(1) applying said filtrate to a first affinity column, wherein said filtrate passes through said first affinity column to produce a flow through and wherein some of said BMP-2 in said filtrate binds to said first affinity column;
(2) eluting said bound BMP-2 from said affinity column by applying a first elution buffer comprising between 1 M and 10 M urea and between 10 mM and 50 mM 3-[cyclohexylamino]-1-propanesulfonic acid (CAPS) buffer, wherein said first elution buffer is at a pH between 8.0 and 12.0;
(3) collecting a first eluent comprising said BMP-2;
(4) applying said first eluent to a size exclusion column to remove said urea and CAPS buffer, wherein said BMP-2 passes through said size exclusion column to produce a second filtrate; and
(5) collecting said second filtrate comprising said BMP-2.

36. The method of claim 35, wherein said method further comprises the steps of:
(6) applying said flow through comprising said BMP-2 to a second affinity column, wherein said BMP-2 binds to said second affinity column;
(7) eluting said BMP-2 from said second affinity column by applying a second elution buffer comprising between 0.1 M and 2 M NaCl, between 10 mM and 1 M Tris-HCl, and between 1 M and 10 M urea, wherein said second elution buffer is at a pH between 5.0 and 10.0;
(8) collecting a second eluent comprising said BMP-2;
(9) applying said second eluent to a size exclusion column to remove said urea, wherein said BMP-2 passes through said size exclusion column to produce a third filtrate; and
(10) collecting said third filtrate comprising said BMP-2.

37. The method of claim 35, wherein said first affinity column is a gelatin-sepharose column.

38. The method of claim 36, wherein said second affinity column is a heparin-sepharose column.

39. The method of claim 35, wherein said urea is present at a concentration of about 4 M, said CAPS buffer is present at a concentration of about 50 mM, and said pH is about 11.0.

40. The method of claim 36, wherein said NaCl is present at a concentration of about 0.7 M, said Tris-HCl is present at a concentration of about 50 mM, said urea is present at a concentration of about 6 M, and said pH is about 7.4.

41. The method of claim 35 or 36, wherein said size exclusion column is a G-100, a G-75, or a G-25 column.

42. The method of claim 35 or 36, wherein after said method, said BMP-2 is greater than 95% pure by weight.

43. The method of claim 24, wherein said BMP-2 is capable of inducing bone formation when administered to a mammal.

44. A method for obtaining greater than 95% pure non-recombinant BMP-2, said method comprising the steps of:
(a) culturing human non-cancer cells in culture medium, wherein said cells express non-recombinant BMP-2; and
(b) purifying said BMP-2 to produce BMP-2 that is greater than 95% pure by weight.

45. The method of claim 44, wherein said human non-cancer cells secrete said BMP-2 into said culture medium.

46. The method of claim 45, wherein said culture medium comprises medium 199.

47. The method of claim 46, wherein said medium further comprises 1.0 to 3.5 g/L bicarbonate salt; 1.0 to 5.0 g/L glucose; 10 to 30 μg/L dexamethasone; 1 to 10 g/L hydrolyzed protein; and 5 to 15 μg/L insulin.

48. The method of claim 47, wherein said medium further comprises an antibiotic.

49. The method of claim 48, wherein said antibiotic is penicillin, which is present at a concentration of 50,000 to 200,000 units/L, and streptomycin, which is present at a concentration of 0.05 to 0.2 g/L.

50. The method of claim 47, wherein said hydrolyzed protein comprises lactalbumin hydrolysate.

51. The method of claim 44, wherein said purification comprises the steps of:
(1) filtering the culture medium, wherein said culture medium comprises BMP-2;
(2) applying said filtrate to a first affinity column, wherein said filtrate passes through said first affinity column to produce a flow through and wherein some of said BMP-2 in said filtrate binds to said first affinity column;
(3) eluting said bound BMP-2 from said affinity column by applying a first elution buffer comprising between 1 M and 10 M urea and between 10 mM and 50 mM 3-[cyclohexylamino]-1-propanesulfonic acid (CAPS) buffer, wherein said first elution buffer is at a pH between 8.0 and 12.0;
(4) collecting a first eluent comprising said BMP-2;
(5) applying said first eluent to a size exclusion column to remove said urea and CAPS buffer, wherein said BMP-2 passes through said size exclusion column to produce a second filtrate; and
(6) collecting said second filtrate comprising said BMP-2.

52. The method of claim 51, wherein said method further comprises the steps of:
(7) applying said flow through comprising said BMP-2 to a second affinity column, wherein said BMP-2 binds to said second affinity column;
(8) eluting said BMP-2 from said second affinity column by applying a second elution buffer comprising between 0.1 M and 2 M NaCl, between 10 mM and 1 M Tris-HCl, and between 1 M and 10 M urea, wherein said second elution buffer is at a pH between 5.0 and 10.0;
(9) collecting a second eluent comprising said BMP-2;
(10) applying said second eluent to a size exclusion column to remove said urea, wherein said BMP-2 passes through said size exclusion column to produce a third filtrate; and
(11) collecting said third filtrate comprising said BMP-2.

53. The method of claim 51, wherein said urea is present at a concentration of about 4 M, said CAPS buffer is present at a concentration of about 50 mM, and said pH is about 11.0.

54. The method of claim 51, wherein said first affinity column is a gelatin-sepharose column.

55. The method of claim 52, wherein said NaCl is present at a concentration of about 0.7 M, said Tris-HCl is present at a concentration of about 50 mM, said urea is present at a concentration of about 6 M, and said pH is about 7.4.

56. The method of claim 52, wherein said second affinity column is a heparin-sepharose column.

57. The method of claim 51 or 52, wherein said size exclusion column is a G-100, a G-75, or a G-25 column.

58. The method of claim 44, wherein said human non-cancer cells are selected from stem cells, macrophages, fibroblasts, vascular cells, osteoblasts, chondroblasts, osteoclasts, and osteocytes.

59. The method of claim 58, wherein said fibroblasts are human fetal lung fibroblasts.

60. The method of claim 59, wherein said human fetal lung fibroblasts are selected from MRC-5 cells and MRC-9 cells.

61. The method of claim 44, wherein said BMP-2 is capable of inducing bone formation when administered to a mammal.

62. A composition comprising non-recombinant human BMP-2, wherein said BMP-2 comprises greater than 95% of said composition and is capable of inducing bone formation when administered to a mammal.

63. A composition for stimulating new bone formation in a patient in need thereof, said composition comprising a pharmaceutically effective dose of a substantially pure non-recombinant BMP-2 polypeptide obtained by the steps of:
(a) culturing human non-cancer cells in culture medium, wherein said BMP-2 polypeptide is secreted into said culture medium; and
(b) purifying said BMP-2 from said culture medium.

64. The composition of claim 62 or 63, wherein said composition further comprises one or more growth factors.

65. The composition of claim 64, wherein said one or more growth factors is selected from the group consisting of insulin-like growth factor (IGF)-I, IGF-II, fibroblast growth factor (FGF), growth hormone (GH), platelet-derived growth factor (PDGF)-I, PDGF-II, interleukin (IL)-1, transforming growth factor (TGF)-α, TGF-β, epidermal growth factor (EGF), tumor necrosis factor (TNF), vascular endothelial growth factor (VEGF), and nerve growth factor (NGF).

66. The composition of claim 62 or 63, wherein said composition further comprises a matrix.

67. The composition of claim 66, wherein said matrix contains one or more of the components selected from the group consisting of fibrin, fibronectin, collagen, gelatin, agarose, a calcium phosphate containing compound, a polymeric particle, and an inorganic filler or particle.

68. The composition of claim 67, wherein said calcium phosphate containing compound is hydroxyapatite, tri-calcium phosphate, or amorphous calcium phosphate.

69. The composition of claim 67, wherein said inorganic filler or particle is ceramic glass, porous ceramic particles or powders, mesh titanium, titanium alloy, particulate titanium, titanium alloy, or bioglass.

70. The composition of claim 67, wherein said polymeric particle is selected from poly(lactic acid), poly(glycolic acid), and copolymers of lactic acid and glycolic acid.

71. The composition of claim 62 and 63, wherein said composition is administered in a solid, paste, gel, or liposome formulation.

72. A method for producing non-recombinant BMP-2, said method comprising the steps of:
(a) culturing mammalian non-cancer cells in culture medium, wherein said cells express and secrete BMP-2 into said culture medium;
(b) separating said culture medium from said cells; and
(c) isolating said BMP-2 from said culture medium.

73. The method of claim 72, wherein said mammalian cells are human cells.

74. The method of claim 72, wherein said mammalian cells are selected from stem cells, macrophages, fibroblasts, vascular cells, osteoblasts, chondroblasts, osteoclasts, and osteocytes.

75. The method of claim 74, wherein said fibroblasts are human fetal lung fibroblasts.

76. The method of claim 75, wherein said human fetal lung fibroblasts are selected from MRC-5 cells and MRC-9 cells.

77. A method for producing non-recombinant BMP-2, said method comprising the steps of:
(a) culturing mammalian non-cancer cells in culture medium, wherein said cells express BMP-2; and
(b) isolating BMP-2 from said culture medium or from said cells in said culture medium.

78. The method of claim 77, wherein said BMP-2 is isolated as an extract of said cells.

79. The method of claim 77, wherein said cells are human cells.

80. The method of claim 77, wherein said cells are selected from stem cells, macrophages, fibroblasts, vascular cells, osteoblasts, chondroblasts, osteoclasts, and osteocytes.

81. The method of claim 80, wherein said fibroblasts are human fetal lung fibroblasts.

82. The method of claim 81, wherein said human fetal lung fibroblasts are selected from MRC-5 cells and MRC-9 cells.

83. A method for stimulating new bone formation in a patient in need thereof, said method comprising administering to said patient a composition comprising a pharmaceutically effective dose of a substantially pure non-recombinant BMP-2 polypeptide obtained by the steps of:
(a) culturing human non-cancer cells in culture medium, wherein said BMP-2 polypeptide is secreted into said culture medium; and
(b) purifying said BMP-2 from said culture medium.
